(19) United States
(12) Patent Application Publication
(10) Pub. No.: US 2004/0007231 A1
(43) Pub. Date: Jan. 15, 2004

(54) Respiratory appliances for pediatric inhalation induction

(75) Inventor: Henry Haifeng Zhou, Englewood Cliffs, NJ (US)

Correspondence Address: HENRY ZHOU, 134 JENKINS DRIVE, ENGLEWOOD CLIFFS, NJ, 07632, US

(73) Assignee: Dr. Henry H. Zhou, Englewood Cliffs, NJ (US)

(21) Appl. No.: 10/064,422

(22) Filed: Jul. 11, 2002

(51) Int. Cl. A61M 15/00; A62B 7/00

(52) U.S. Cl. 128/202.16; 128/206.21

(57) ABSTRACT

The present invention is a respiratory appliance (a face mask or a adaptor of a breathing circuit) with at least one toy, which is played by blowing. The respiratory appliance is used to connect to a breathing circuit and is suitable for pediatric inhalation induction of anesthesia. The respiratory appliance contains adjustable one way valves which control air flows for breathing in anesthetic agents and blowing the toy. Children can smoothly go to sleep when they play with the toy by blowing inside the mask.

### BACKGROUND OF INVENTION

This invention relates to respiratory appliances which can please a child in an operating room and provide a smooth pediatric inhalation induction for general anesthesia.

General anesthesia for children is usually induced by an inhalation technique which involves administering anesthetic gas to a child through a breathing circuit and a face mask (U.S. Pat. Nos. 3,982,532; 4,449,526; 5,429,683 and 5,884,624). During inhalation induction, the mask is put over the child's face, covering both nose and mouth. After anesthetic gas is turned on, the child breathes in anesthetic agents inside the mask and goes to sleep. To prevent air leaks, the mask has to be placed tight on patient's face.

The main disadvantage of this technique is uncomfortable and frightening for most children. Children often feel scared in an operating room. Fear is getting worse when a mask is placed tight over their face. They often fight to resist the mask, that may cause some complications such as aspiration and laryngospasm, and makes the inhalation induction more difficult for an anesthesiologist. A bad experience can make the child become claustrophobic in the rest of his/her life. Even after oral premedication with sedatives such as midazolam, fighting with face mask is still often seen.

In view of the foregoing, it is important to have a smooth inhalation induction. To reach this goal, a face mask which is attractive for kids would be an advancement.

### SUMMARY OF INVENTION

The object of the present invention is to provide a face mask for pediatric inhalation induction. The face mask contains at least one toy which is played by blowing or breathing, such as a balloon or a blow-out. The mask is connected to a breathing circuit and an anesthesia machine. Kids will feel comfortable and have fun to blow the toy via the mask. At the same time, they will inhale anesthetic agents and go to sleep after several deep breaths. Thus, this invention can provide a smooth inhalation induction of anesthesia for children.

### BRIEF DESCRIPTION OF DRAWINGS

FIG. 1 shows a perspective view of a face mask with a balloon;

FIG. 2 shows a bottom view of the mask;

FIG. 3 is a sectional view taken on the line 3,5-3,5 of FIG. 1 showing a one way valve during mask induction;

FIG. 4 shows an enlarged sectional view taken from a portion circled and labeled as “4” in FIG. 3;

FIG. 5 is a sectional view taken on the line 3,5-3,5 of FIG. 1 showing the one way valve adjusted after induction;

FIG. 6 shows an enlarged sectional view taken from a portion circled and labeled as “6” in FIG. 5;

FIG. 7 shows a perspective view of a face mask with a blow-out (before blowing);

FIG. 8 shows a perspective view of a face mask with a blow-out (after blowing);

FIG. 9 shows a sectional view taken on the line 9-9 of FIG. 7;

FIG. 10 shows an enlarged view taken from a portion circled and labeled as “10” in FIG. 9;

FIG. 11 shows a perspective view of an adaptor;

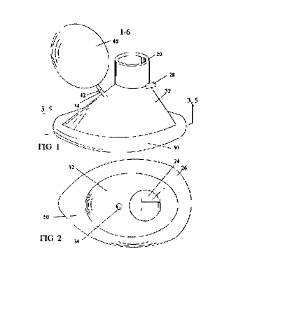

FIG. 1 2 shows a sectional view taken on the line 12-12 of FIG. 11;

FIG. 13 shows an enlarged view taken from a portion circled and labeled as “13” in FIG. 12.

### DETAILED DESCRIPTION

FIGS. 1-6: Preferred Embodiment

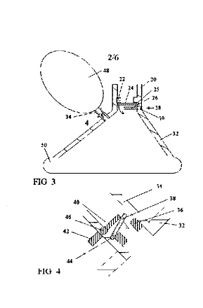

Perspective views of the present invention are illustrated in FIG. 1 and FIG. 2 (bottom view). At the top of a mask (FIG. 1) there is an opening or tube 20. The opening 20 is used to connect to a breathing circuit. At the bottom of the mask, there is a cushion 50 to facilitate the sealing between the mask and a patient's face. To distinguish this invention from previous inventions, this mask has at least one small opening or tube 34 at body 32 of the mask. The tube 34 is connected to a balloon 48 or another toy which is played by blowing. To control air flow between mask and the balloon 48, the opening 20 and the tube 34 contain adjustable one way valves.

Cross sectional views (FIG. 3-FIG. 6) show structures and functions of adjustable one way valves in the mask. At the opening 20 (FIG. 3,5), an one way valve consists of a narrow inside opening 22, a plate or valve 24, a support or holder 26 and a switch or lever 28 which controls the position of the support 26. The support 26 is an “U” shape structure (FIG. 2) supporting the position of the valve 24 (FIG. 2,3). The valve 24 and the support 26 have an axle 25 and 30 inserted in the wall of the opening 20.

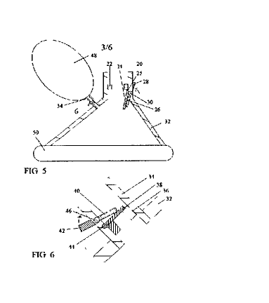

Therefore the valve 24 and the support 26 can be rotated up and down. The structure of one way valve in the tube 34 is similar to that in the opening 20. In the enlarged view (FIG. 4), the tube 34 consists of a narrow inside opening 36, a plate or valve 38, an “U” shape support or holder 40 and a switch or lever 42 controlling the position of the support 40. The valve 38 and the holder 40 have an axle 44 and 46 inserted in the wall of the tube 34.

During induction of anesthesia, a child lies on his/her back at an operating table. The opening 20 of the mask is connected to a breathing circuit. The cushion 50 of the mask is placed over the child's face, covering both nose and mouth. When the child breathes in, negative air pressure inside the mask will pull the valve 24 down to the support 26 as indicated by an arrow (FIG. 3) and the one way valve is opened. Thus the child will inhale anesthetic agents from the breathing circuit. On the other hand, the negative pressure inside the mask will pull the valve 38 down as indicated by an arrow (FIG. 4) and close the opening 36. Therefore the child will not re-breathe air from the balloon 48. When the child breathes out, positive air pressure in the mask will push the valve 24 up against the opening 22 and close the one way valve. On the other hand, air in the mask will push the valve 38 up to the holder 40, open the opening 36 and blow up the balloon 48.

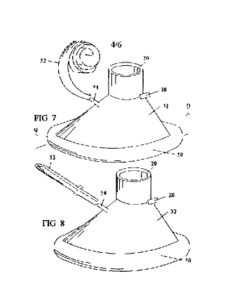

After several deep breaths, the child will be induced to sleep. At this time, blowing balloon become unnecessary. The one way valve of opening 20 can be opened (FIG. 5) by pushing the lever 28 up (as indicated by an arrow) and turning the support 26 down. The valve 24 will fall down with the support 26 by gravity. Therefore positive pressure in the mask can not push the valve 24 up to close the opening 22. The opening 20 will remain open during both inspiration and expiration. On the other hand, the opening 36 to the balloon 48 should be closed after the child sleeps. Turning the lever 42 up as indicated by an arrow (FIG. 6) and pushing the holder 40 down will press the valve 38 against the opening 36 and prevent air from getting into the balloon 48.

FIGS. 7-10: Alternative Embodiment #1

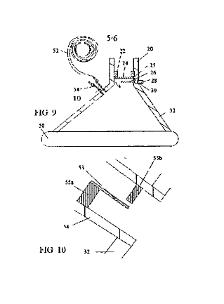

There are various possible toys which can be played by blowing with the mask. FIGS. 7-8 (perspective view) and 9-10 (sectional view) show a mask with a blow-out, a paper tube for blowing and producing musical or whistling sound. The perspective views show blow-out before (FIG. 7) and after (FIG. 8) blowing. Similar to the mask with a balloon in FIG. 1, the mask has an opening or tube 20, which is used to connect to a breathing circuit, and a cushion 50 at the bottom of the mask. The opening 20 also contains an adjustable one way valve. To distinguish this invention from previous inventions, this mask has at least one small opening or tube 54 at body 32. The tube 54 is connected to a blow-out 52.

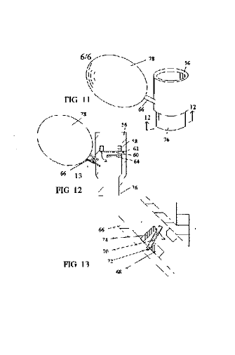

The one way valve in the opening 20 consists of a narrow inside opening 22, a plate or valve 24, a support or holder 26 and a switch or lever 28 which controls the position of the support 26 (FIG. 9). The support 26 is an “U” shape structure supporting the position of the valve 24. The valve 24 and the support 26 have an axle 25 and 30 inserted in the wall of the opening 20. Therefore the valve 24 and the support 26 can be rotated up and down. The tube 54, connecting between the mask and the blow-out 52, has narrow regions or members 55a and 55b (FIG. 10). There is a thin plate 53 extended from the region 55a to region 55b. The plate 53 and the region 55b form a narrow opening in tube 54.

During induction of anesthesia, the opening 20 of the mask is connected to a breathing circuit. The cushion 50 of the mask is placed over a child's face. When the child breathes in, negative air pressure inside the mask will pull the valve 24 down to the support 26 as indicated by an arrow (FIG. 9) and open the one way valve. Thus the child inhales anesthetic agents from the breathing circuit. When the child breathes out, positive air pressure in the mask will push the valve 24 up against the opening 22 and close the one way valve. Instead air in the mask will pass the narrow opening between plate 53 and region 55b in tube 54 (FIG. 10) and blow the blow-out 52 (FIG. 8). Blowing air through the narrow opening at the plate 53 and region 55b can also produce musical or whistling sounds.

After the child is induced to sleep, blowing blow-out 52 become unnecessary. The one way valve of opening 20 can be opened (FIG. 9) by pushing the lever 28 up and turning the support 26 down. The valve 24 will fall down with the support 26 by gravity. Therefore positive pressure in the mask can not push the valve 24 up to close the opening 22. The opening 20 will remain open during both inspiration and expiration.

FIGS. 11-13 Alternative Embodiment #2

The present invention can be presented as an adaptor or a pipe with a balloon showed by FIG. 11 (perspective view) and 12 (sectional view). The adaptor has a top end opening 56, which connects to a breathing circuit, and a bottom end opening 76, connecting to a mask or a mouthpiece for breathing. To distinguish this invention from previous airway adaptor, this adaptor has at least one small opening or tube 66, which connected to a balloon 78 or another toy, and has one way valves in the opening 56 and the tube 66.

Cross sectional views (FIG. 12, 13) show structures and functions of one way valves in the adaptor. At the opening 56 (FIG. 12), an one way valve consists of a narrow inside opening 58, a plate or valve 60 and a support or holder 64. The support 64 is an “U” shape structure supporting the position of the valve 60. The valve 60 has an axle 62 inserted in the wall of the opening 56. Therefore the valve 60 can rotated up and down. The structure of one way valve in the tube 66 is similar to that in the opening 56. In the enlarged view (FIG. 13), the tube 66 consists of a narrow inside opening 68, a plate or valve 70 and an “U” shape support or holder 74. The valve 70 has an axle 72 inserted in the wall of the tube 66.

Before induction of anesthesia, the adaptor is connected to the breathing circuit and a regular mask or a mouthpiece. When the child breathes in, negative pressure inside the mask will pull the valve 60 down to the support 64 as indicated by an arrow (FIG. 12) and the one way valve is opened. Thus the child will inhale anesthetic agents from the breathing circuit. On the other hand, the negative pressure will pull the valve 70 down as indicated by an arrow (FIG. 13) and close the opening 68. Therefore the child will not re-breathe air in the balloon 78. When the child breathes out, positive pressure will push the valve 60 up against the opening 58 and the one way valve is closed. Instead air can push the valve 70 up to the holder 74, open the opening 68 and blow up the balloon 78. After the child is induced to sleep, blowing balloon become unnecessary. The adaptor can be disconnected or removed from the breathing circuit.

I claim:

1. A respiratory appliance for pediatric inhalation induction comprising:
(a) a face mask having a wall, a cushion at a lower edge of said wall and an opening connecting to a respiratory circuit, and
(b) at least one toy, which is played by blowing or breathing, each connecting to one tube which joins to said wall of said mask by means for providing an airway from said mask to said toy;
whereby a child can play said toy by breathing inside said mask during said inhalation induction.

2. A respiratory appliance as claimed in claim 1, wherein said opening of said mask and said tube further include an one way valve which can be adjusted to an complete open or close status by means for controlling airflow from said mask to said toy.

3. A respiratory appliance for pediatric inhalation induction comprising:
(a) an adaptor or pipe having a upper opening, containing an one way valve, connecting to a respiratory circuit and a lower opening connecting to a hollow member which is contiguous to a patient's face by means for breathing, and
(b) at least one toy, which is played by blowing or breathing, each connecting to one tube which contains an one way valve and joins to a wall of said adaptor by means for providing an airway from said adaptor to said toy;
whereby a child can play said toy by breathing with said adaptor during said inhalation induction.

4. A respiratory appliance as claimed in claim 1 and 3, wherein said toy includes balloons, inflatable toys, blow-outs, a device making musical or whistling sounds by means of breathing and a toy producing movement by means of breathing.
